(19) United States
(12) Patent Application Publication
(10) Pub. No.: US 2011/0160522 A1
(43) Pub. Date: Jun. 30, 2011

(54) ELF HOMEOPATHIC CHRONOMETER

(76) Inventor: Daniel D. Brunda, Ewing, NJ (US)

(21) Appl. No.: 12/650,634

(22) Filed: Dec. 31, 2009

(51) Int. Cl. A61M 21/00

(52) U.S. Cl. 600/26

(57) ABSTRACT

Provided is a portable chronometer including a chronometer body for encasing a chronometer mechanism and an electromagnetic frequency (EMF) generator. The EMF generator is controlled by a switch to output a desirable EMF signal.

### FIELD OF THE INVENTION

This invention generally relates to the field of electric crystal frequency watches, also known as chronometers, and, more particularly, to special scientific frequencies which have been found to benefit human beings, as well as those which are required to nullify the frequencies normally encountered by human beings in the environment.

### BACKGROUND OF THE INVENTION

Philip Stein™ offers watches which utilize frequency based technology and address the natural frequency of the Earth, approximately 7-9 Hz. Therapists and healers consider this to be the frequency at which the human body functions most efficiently. In one technology, a Philip Stein™ watch replicates the Earth's natural frequency of 7-9 Hz, at which many wearers report positive benefits and an overall improvement in their well being.

Philip Stein™ has also introduced multiple frequency technology (MFT) in their Prestige™ collection of watches. Under this technology a watch is permanently programmed with multiple natural well being frequencies in addition to the 7-9 Hz Earth frequency. It is believed that both technologies help to balance the body's energy flow. Philip Stein™ customers are thus able to select the frequency which works best for them. The exact frequency output is most precisely determined by the use of a signal generator and a spectrum analyzer. For an example of such a measurement, see U.S. Pat. No. 5,350,999, issued to Brunda, which is incorporated herein by reference.

However, the natural frequency of the human heart is approximately 1.0 Hz. See FIG. 1. There are also several other extremely low frequency (“ELF”) environmental frequencies which affect the well being of human beings, such as 4 Hz, 16 Hz, 50 Hz, 60 Hz and 150 Hz. See, for example, Power Line Radiation, Your Genes, Hereditary Diseases, The Unified Nature Of Electromagnetic Radiation, Energy And Control And The Radiation Limits Of Human Beings, by Daniel Brunda, Table 7, page 3 (Xlibris Corporation, Book No. 19987, ISBN: 1-4134-3084-8.)

As a human being moves throughout the day, it will inevitably move between areas having different ambient electromagnetic frequencies (“EMF”). Having a watch or chronometer that constantly emits a fixed frequency (or frequencies) will not always result in the most efficient transmission of EMF to the user because of these changing ambient conditions.

Thus, there is a need for a portable device which can provide healthful EMF frequencies wherever the user travels.

Moreover, there is a need for a portable device that allows a user to select from a predetermined assortment of electromagnetic frequencies dependent on personal preference.

There is also a need for a portable device that can be programmed by a user to output EMF at one or more frequencies input by the user.

### SUMMARY OF THE INVENTION

An aspect of the present invention provides A portable chronometer having a chronometer body for encasing a chronometer mechanism and an electromagnetic frequency (EMF) generator. The EMF generator outputs an EMF signal.

In another aspect of the present invention, the portable chronometer also includes a switch for operating the EMF generator, the switch providing an “on” and an “off” selection, enabling and disabling EMF signal output by the EMF generator, respectively.

In another aspect of the present invention, the EMF generator is further configured with a predetermined list of one or more desirable frequencies, and a control means is provided for selecting one or more of the desirable frequencies for the EMF generator to output. In another aspect, the control means is configured to accept user input of one or more frequencies for the EMF generator to output. The processor and memory may also be further configured to retrievably store the input of one or more desirable frequencies.

### BRIEF DESCRIPTION OF THE DRAWINGS

FIG. 1 is a depiction of the portable chronometer, in accordance with an embodiment of the present invention;

FIG. 2 is a schematic diagram of the EMF generator portion of a chronometer system, in accordance with an embodiment of the present invention;

FIG. 3a is a flowchart of the process of a user selecting a frequency to output, in accordance with an embodiment of the present invention; and

FIG. 3b is a flowchart of a user inputting a frequency to output, in accordance with an embodiment of the present invention.

### DETAILED DESCRIPTION

In the following description, for purposes of explanation, specific numbers, materials and configurations are set forth in order to provide a thorough understanding of the invention. It will be apparent, however, to one having ordinary skill in the art, that the invention may be practiced without these specific details. In some instances, well-known features may be omitted or simplified so as not to obscure the present invention. Furthermore, reference in the specification to “one embodiment” or “an embodiment” means that a particular feature, structure or characteristic described in connection with the embodiment is included in at least one embodiment of the invention. The appearances of the phrase “in an embodiment” in various places in the specification are not necessarily all referring to the same embodiment.

The present invention advantageously provides for a portable device, such as a chronometer having an EMF generator able to generate an EMF. In an embodiment the present invention also provides a portable device that allows a user to select from a predetermined assortment of electromagnetic frequencies dependent on personal preference. Also in an embodiment, the present invention provides a portable device that can be programmed by a user to output EMF at one or more frequencies which are input to the device by the user.

The present invention takes advantage of the fact that in order to obtain optimal wellness and natural healing of the human body, particular electromagnetic frequencies are desirable. For an example of a desirable frequency, the human heart operates at approximately 1.0 Hz. Also, the Earth's natural frequency is about 8 Hz. These frequencies have been demonstrated to benefit the health and welfare of human beings.

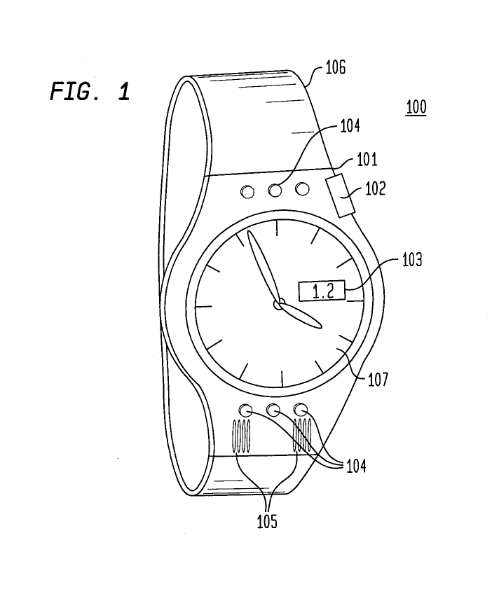

FIG. 1 depicts an exemplary portable chronometer 100. In an embodiment of the present invention, the chronometer 100 includes a wristband 106 and chronometer body 101. As is understood to those of ordinary skill in the art, the wristband 106 may be made of metal, plastic, leather or other suitable material, and likewise may be capable of expansion to fit around different-sized wrists. Also, it is understood that the chronometer body 101 may be composed of metal, plastic or other suitable material.

Choice of the construction materials is also dependent on the method of transmission of EMF energy to the body desired. For example, if the EMF energy is to be transmitted to the body by conduction, metals are preferable for use in constructing the chronometer. Alternatively, if the EMF energy is to be transmitted to the body by induction, non-metal construction materials are preferred. If the transmission id to be achieved by both induction and conduction, then a combination of metals and non-metals are preferred.

The chronometer 100 also includes a watch face 107 for providing a time measurement to a user. The watch face 107 may be digital (not depicted) or use the more traditional minute and hour hands with time increments depicted along the watch face's 107 border. It is presumed that the time measurement on the watch face 107 is controlled by a clock mechanism and set by a control mechanism such as is well known to those of ordinary skill in the relevant art, and details of these features are intentionally not depicted in the Figures for clarity.

Also depicted in FIG. 1 are control means, such as control buttons 104. It is intended that various embodiments of the present invention provide a variety of control means, with the control buttons 104 depicted here being exemplary. For example, the control means may alternatively consist of slider controls, dial controls, or other switching means, or combinations of these elements. The control means are used in the chronometer 100 to determine whether or not and, if so, what EMF frequency or frequency the chronometer 100 should output. Also included in the chronometer is a display 103 for displaying the EMF frequency being output. This display 103 may also be provided in several formats, e.g., LED, LCD, etc.

Another feature of the exemplary chronometer 100 of FIG. 1 is the EMF frequency output area 105. It is intended that the EMF generator output EMF radiation at least through the output area 105, regardless of any EMF shielding provided by the chronometer body 101.

Also provided in FIG. 1 is a USB 2.0 port 102 for optionally connecting an external programming device to the chronometer's internal processor, which is also described herein. In an embodiment of the invention it is useful for the chronometer to be programmed externally via a USB connection with an external computer. In this way, new or different EMF frequencies may be downloaded onto the chronometer 100 for output. In addition, diagnostics may be performed on the chronometer's components. Although a USB 2.0 port is provided in FIG. 1, it is understood that any communication channel that can be provided in or on a chronometer body 101 may be used.

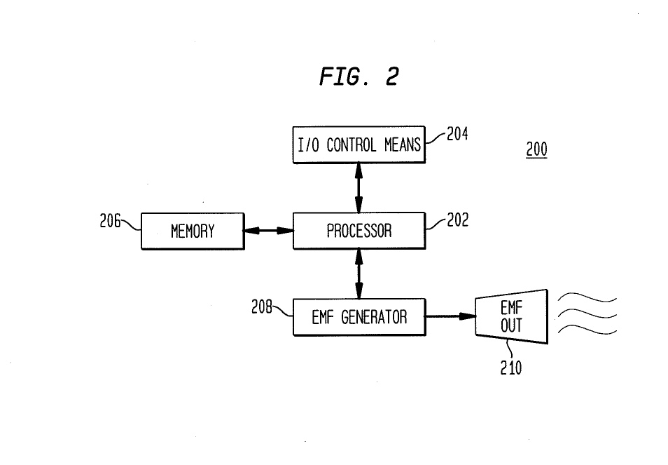

FIG. 2 depicts a schematic diagram of the portion of an exemplary chronometer system used for EMF generation. In an embodiment of the present invention, the system 200 includes a I/O control means, such as control buttons 104, in communication with a processor 202. An external programming interface, such as a USB 2.0 port may also be included as part of the I/O control means 204.

The processor 202 is further in communication with associated electronic memory 206, and controls an EMF generator 208, which outputs the EMF generation signal to an EMF output device 210. Each of the aforementioned components operates using electrical power from a battery (not depicted). Processor 202 may be any general purpose microprocessor configured with operating software allowing the processor 202 to manage, control and accept input from the other devices with which it communicates. Alternatively, processor 202 may be a special-purpose processor designed to optimize power consumption and other features advantageous in a small device.

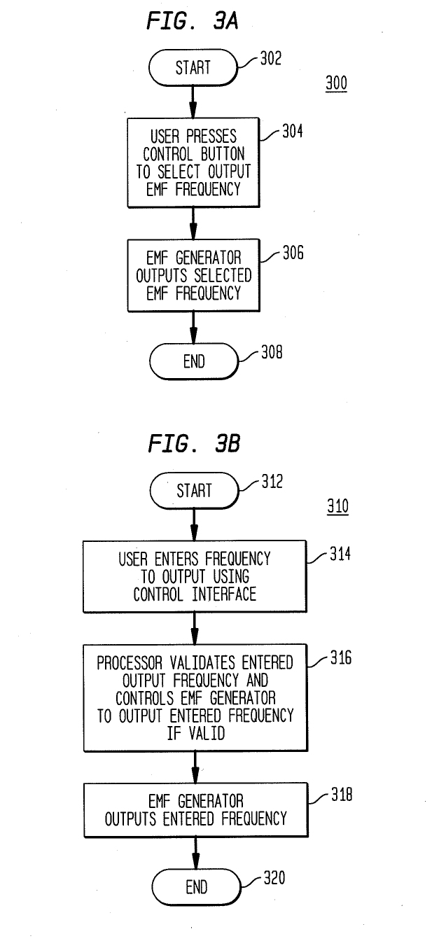

FIG. 3a depicts a flowchart of an exemplary process of a user selecting a frequency to output. In an embodiment of the invention, the process starts 302 when the user presses or selects a control button to select an output EMF frequency 304. next, the EMF generator outputs the selected EMF frequency 306 and the process is complete 308.

FIG. 3b depicts a flowchart of an exemplary process of a user inputting a frequency to output. In an exemplary embodiment of the invention, the user starts 312 by entering a frequency to output using the control interface means 314. Next, the processor validates that the entered output frequency can be generated by the EMF generator, and, if so, controls the EMF generator to output the entered frequency 316. If the entered frequency cannot be generated by the EMF generator, the processor can indicate this by signaling the user (step not depicted). If the entered frequency can be output, the EMF generated then outputs the entered frequency 318, and the process is complete 320.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A portable chronometer comprising:
a chronometer body for encasing:
a chronometer mechanism; and
an electromagnetic frequency (EMF) generator;
the EMF generator configured to output a desirable EMF signal.

2. The chronometer according to claim 1, further comprising a switch for operating the EMF generator,
wherein the switch provides an “on” and an “off” selection, enabling and disabling EMF signal output by the EMF generator, respectively.

3. The chronometer according to claim 1, wherein the EMF generator is further configured with a predetermined list of one or more desirable frequencies, and a control means is provided for selecting one or more of the desirable frequencies for the EMF generator to output.

4. The chronometer according to claim 1, wherein the EMF generator is further configured to accept input of one or more desirable frequencies to output from an input control means.

5. The chronometer according to claim 4, further comprising a computer processor and associated electronic memory in controlling communication with the EMF generator and an input control means, the processor and memory configured to accept input of one or more desirable frequencies and control the EMF generator to output the one or more desirable frequencies.

6. The chronometer according to claim 5, wherein the processor and memory are further configured to retrievably store the input of one or more desirable frequencies.
